(19) United States
(12) Patent Application Publication
(10) Pub. No.: US 2026/0131028 A1
(43) Pub. Date: May 14, 2026

(54) COMBINATION CANCER THERAPY

(71) Applicant: DELTA NEXT-GENE, LLC, Santa Monica, CA (US)

(72) Inventors: Erlinda M. Gordon, Santa Monica, CA (US); Fredrick L. Hall, Santa Monica, CA (US)

(21) Appl. No.: 18/855,295

(22) PCT Filed: Apr. 7, 2023
(86) PCT No.: PCT/US2023/017925

§ 371 Date: Oct. 8, 2024

Related U.S. Application Data
(60) Provisional application No. 63/329,177, filed on Apr. 8, 2022.

(51) Int. Cl. A61K 48/00; A61K 38/17; A61K 39/395; A61K 40/15; A61P 35/00; C12N 15/86

(52) U.S. Cl. CPC A61K 48/0058; A61K 38/17; A61K 39/3955; A61K 40/15; A61P 35/00; C12N 15/86; C12N 2740/13043

(57) ABSTRACT

The present disclosure relates to methods of coupling antic-cancer agents and anti-cancer treatments to improve the efficacy of the overall treatment. The anti-cancer agent reduces stroma in the tumor microenvironment, in part by killing stroma producing cells such as TAFs and TAMs. Reduction of tumor stroma not only improves immune cell function, including immune cells administered in a second treatment, but also allows allowing other anti-cancer treatments to better access to tumor cells.

### CROSS-REFERENCES TO RELATED APPLICATIONS

This application claims priority to U.S. Provisional Patent Application No. 63/329,177, filed Apr. 8, 2022, and titled “COMBINATION CANCER THERAPY,” the contents of which are incorporated herein in their entirety.

### SEQUENCE LISTING

The instant application contains a Sequence Listing which has been submitted electronically in XML format and is hereby incorporated by reference in its entirety.

### BACKGROUND

Cancer is a major public health and economic issue and the burden of this disease is expected to grow. 18 million cases of cancer were diagnosed in 2018, and this number is expected to reach 29 million by 2040, due to aging and growth of the population. Moreover, an estimated 43.8 million people were living with cancer in 2018, having been diagnosed in the previous five years.

Early treatments for cancer involved the use of chemicals (chemotherapy) that non-specifically killed rapidly dividing cells. While such treatments would kill cancer cells, they also caused discomforting short-term side effects, such as nausea and hair loss, and serious long-term side effects such as heart damage, nerve damage and fertility problems. Moreover, cancer cells often became resistant to chemotherapeutic agents.

In recent decades, developments in biotechnology have led to breakthrough, biology-based treatments for cancer. The advent of precision medicine exemplified by tyrosine kinase inhibitors, and the renaissance of immunotherapy such as NK cell therapy, immune checkpoint and cell cycle checkpoint (CDK) inhibitors and targeted gene therapies have revolutionized the practice of cancer medicine away from toxic chemotherapy. To date, there are at least 12 clinical trials involving expanded allogeneic or autologous NK cells with or without an immune checkpoint inhibitor, monoclonal antibodies, chemotherapy/radiation therapy for various cancer types including medulloblastoma, colon cancer, HER2 positive cancers, lymphoma, myeloid leukemia, MDS, and multiple myeloma. Furthermore, the combination of immune checkpoint inhibitors with NK cell-based therapeutic strategies aim to strengthen its efficacy as an antitumor therapy.

Many of these therapies involve recruiting T cells and other immune cells to antigens expressed on the tumor calls, which results in the immune system attacking the tumor cells.

While immunotherapy has had success in treating cancer, there are many problems that remain to be solved. One such problem involves the tumor stroma. Tumor stroma is broadly defined as the non-cancer cell and non-immune cell component of tumors. Tumor stroma is composed of extracellular matrix and specialized connective tissue cells, including fibroblasts and mesenchymal stromal cells. All tumors have stroma and in fact it is necessary for nutritional support of the tumor and removal of waste products. While aiding the tumor in these ways, stroma has also been implicated in resistance to multiple types of cancer therapy. Moreover, the existence of certain tumor-associated fibroblasts (TAFs), a major type of stromal cell in tumors, can be predictive of cancer recurrence after chemotherapy treatment. A significant barrier to developing broadly effective immunotherapies against solid tumors has been the inability to sustain cytotoxic and proinflammatory immune cell functions in the tumor microenvironment.

One way that stroma causes resistance to therapy is by physically shielding tumor cells from treatment. For example, a dense stromal layer may prevent agents, such as chemotherapeutic agents and biological agents (e.g., checkpoint inhibitors) from reaching tumor cells. Likewise, targeted therapies that utilize cell surface receptors or antigens, such as receptor target drugs, T-cells, etc., are blocked by stroma from reaching their receptors or antigens.

In addition, NK cell-associated cytotoxicity can be impaired by stromal cells, for example cancer-associated fibroblasts, monocytes, macrophages and other immune cells. Fibroblasts in the tumor microenvironment (TME) suppress the function of NK cells through downregulating ligands of NK cell activating receptors, enhancing tumor-associated macrophages enrichment, and extracellular matrix (ECM) production such as IDO and PGE2.

Mechanistically, improved efficacy may be achieved by combining SNKO1 and an immune checkpoint inhibitor with targeted cell cycle checkpoint inhibitor, e.g., DeltaRex-G, a tumor targeted retro vector (1) that hunts down tumors wherever they are and nowhere else, and (2) that kills not only cancer cells/cancer stem cells, but also proliferative tumor associated microvasculature (TAMs) and tumor associated fibroblasts (TAFs) that make the stroma that downregulate ligands of NK cell activating receptors and prevent immune cell trafficking in the TME. Killing TAFs, proliferative cells, and reducing ECM production by DeltaRex-G therapy will enable NK cell and other immune cell entry in the TME and favor NK cell-ligand interactions for improved tumor control.

Thus, reducing or eliminating tumor stroma would have beneficial effects on treatment. The present application describes a method of coupling anti-cancer treatments with agents that reduce or eliminate tumor stroma, thereby improving the efficacy of the anti-cancer treatments.

### DETAILED DESCRIPTION

The present disclosure relates to a method of improving anticancer treatments. More specifically, the present disclosure relates to coupling anticancer treatments with agents that reduce or eliminate tumor stromal. Reduction or elimination of tumor stroma allows other therapeutic agents to more easily access tumor cells. Thus, an invention of the disclosure may generally be practiced by administering to an individual an anti-cancer agent that reduces or eliminates tumor stroma, and/or stroma producing cells, such as TAFs and tumor-associated macrophages (TAMs), and then administering to the individual a second anti-cancer treatment. Examples of second anti-cancer treatments include, but are not limited to, administering immune checkpoint inhibitors, administering T-CELLS, including CAR-T cells, and administering NK cells.

The extracellular matrix (ECM) plays an important role in cancer progression. It can be divided into the basement membrane (BM) that supports epithelial/endothelial cell behavior and the interstitial matrix (IM) that supports the underlying stromal compartment. Collagens are a major component of the ECM. Breaching of the BM and turnover of e.g., type IV collagen, is a well described part of tumorigenesis. The IM is dominated by the fibrillar-forming collagens type I, II, III, V, XI, XXIV, XXVII and the beaded filament type VI collagen synthesized by the fibroblasts residing in the stroma.

During cancer, the ECM-dynamics are skewed. It is well established that cancer cells secrete high amounts of MMPs, which in turn remodel and degrade the BM. The remodeling of the BM leads to a complex chaos of pro- and antitumor signals from degradation products. The role of type IV collagen turnover, within the BM, has been extensively studied in relation to tumor biology. Several studies have shown that proteolytic cleavage of collagen IV can expose so-called cryptic domains, which are normally hidden when collagen IV is fully assembled. Similar things have been seen with other BM collagens e.g., type XVIII collagen. Depending on the context, these cryptic sites have both pro- and anti-tumor effects; still the turnover and degradation of BM collagens are intrinsically associated with the invasive phenotype of malignant cells. Sites on collagen referred to as signature (SIG) elements, may be used to target anti-cancer agents to stroma, including TAFs and TAMs.

One aspect of the disclosure is method of treating cancer in an individual, the method comprising administering to the patient: i) an anti-cancer agent comprising a binding peptide configured to bind one or more signature (SIG) elements of a tumor; and, ii) at least one anti-cancer treatment comprising cancer immunotherapy.

In such aspect, the anti-cancer agent is targeted to tumor stroma and/or TAFs and/or TAMs, and particularly to SIG elements present in collagen. In targeting the stroma, and in particular TAFs and TAMs, the anti-cancer agent delivers a toxic payload to the cells, thereby killing the TAFs and/or TAMs, resulting in decreased production of stroma. This allows improved functioning of immune cells, including those delivered in an anti-cancer treatment, and also allows compounds delivered in an anti-cancer treatment, such as immune checkpoint inhibitors, to better access tumor cells.

In one aspect the anti-cancer agent comprises a cyclin G1 inhibitor. Delivery of a cyclin G1 inhibitor to a cell results in the inhibition of cyclin G1, which results in cell death. The cyclin G1 inhibitor may comprise a dominant negative cyclin G1 mutant, which may be dnG1. The cyclin G1 inhibitor comprises a truncated form of the cyclin G1 protein, which may lack the ubiquitinated N-terminus and the α1 and a2 helical segments of native cyclin G1. The truncated form of the cyclin G1 protein may amino acids 1-86 of the full-length cyclin, and thus may consist of amino acids 87-295 of full-length cyclin G1. The anti-cancer agent may comprise a nucleic acid molecule, which may be a recombinant viral vector, encoding the cyclin G1 inhibitor.

In methods of the disclosure, the binding peptide may bind to a SIG element on collagen. The binding peptide may comprise an amino acid sequence that binds to a SIG element on collagen. The binding peptide may be configured to bind a Gly-Xxx-Pro/Hyp-Ala-Xxx-Pro/Hyp-Gly-Xxx-Pro/Hyp polypeptide sequence that is exposed on a tumor, wherein Xxx in position 2, 5 and 8 is an amino acid other than Gly, Pro, or Hyp, and Pro/Hyp in position 3, 6 and 9 means either Pro or Hyp, for example or Gly-Xxx-Pro-Ala-Xxx-Pro/Hyp-Gly-Xxx-Pro/Hyp (SEQ ID NO:3). The binding element may comprise an amino acid sequence selected from the group consisting of Trp-Arg-Glu-Pro-Ser-Phe-Met-Ala-Leu-Ser (SEQ ID NO:1) and Trp-Arg-Glu-Pro-Gly-Arg-Met-Glu-Leu-Asn (SEQ ID NO:2). In some aspects, the anti-cancer agent may comprise a nanoparticle, or a viral or pseudoviral particle, and the binding peptide is displayed on the surface of the nanoparticle, or viral or pseudoviral particle.

In methods of the disclosure, the anti-cancer treatment may comprise cancer immunotherapy, which may comprise administering immune cells to the individual. The immune cells may comprise NK cells, super NK cells, activated NK cells, T cells, CAR-T cells, and/or modified or engineered cells thereof.

The anti-cancer treatment may comprise cancer immunotherapy, which may comprise administering immune checkpoint inhibitors or monoclonal antibodies.

In some aspects, the anti-cancer agent may comprise a thymidine kinase (TK) gene. In some aspects, the anti-cancer treatment may comprise cancer immunotherapy, which may comprise administering a cytocidal compounds that is activated by TK. The cytocidal compound may be a synthetic nucleoside analogue, which may be acyclovir or valacyclovir.

In some aspects, the anti-cancer agent may be DeltaRex-G or Delta-RexGT. Construction and use of Delta-RexG and DeltaRex-GT are disclosed in U.S. Patent Publication US2019/0382459, which is incorporated herein by reference in its entirety (see, in particular, FIG. 3 and corresponding text).

In methods of the disclosure, the anti-cancer agent and the anticancer treatment may, but need not, be administered to the individual at separate times. The anti-cancer agent may be administered at a first time, and the anti-cancer treatment may be administered at a later, second time. In one aspect, the anti-cancer treatment may be administered at a first time, and the anti-cancer agent may be administered at a later, second time. In some aspects, the second time may be at least 15 minutes, at least 30 minutes, at least 1 hour, at least hours, at least 12 hours, at least 24 hours, at least 2 days, at least 3 days, at least 4 days, at least 5 days, at least one week, at least two weeks, or at least one month, after the first time.

One aspect of the disclosure is a kit comprising an anti-cancer agent of the disclosure and components for practicing an anti-cancer treatment of the disclosure. The kit may comprise other components, such as needle, syringes, tubes, vials, and instructions for using an anti-cancer agent of the invention in conjunction with an anti-cancer treatment to practice a method of the disclosure.

This written description uses examples to disclose the invention, including the best mode, and also to enable any person skilled in the art to practice the invention, including making and using any devices or systems and performing any incorporated methods. The patentable scope of the invention is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal languages of the claims.

Additionally, the contents of US20190382459, entitled “CYCLIN G1 INHIBITORS AND RELATED METHODS OF TREATING CANCER,” and appended to this document, are incorporated herein by reference.

### EXAMPLES

Background and Rationale: Pancreatic cancer is the fourth leading cause of death affecting −42,000 persons in the United States, with less than 5% 5-year survival rate for patients with Stage 4 disease. Therefore, innovative therapies are urgently needed. DeltaRex-G is an “off-the-shelf” tumor-targeted gene vector that (a) displays a Signature (5ZG)-binding peptide on its surface for targeting the tumor microenvironment (TME), and (b) encodes a cytocidal cyclin G1 (CCNG1) inhibitor gene for eradicating cancer cells, tumor associated microvasculature (TAMs), tumor associated fibroblasts (TAFs) and cancer stem cells. When injected intravenously, the DeltaRex-G nanoparticles seek out and accumulate in the cancerous lesions where Signature (SIG) proteins are abnormally found, in the vicinity of cancer cells, hence augmenting effective drug concentration without collateral damage to normal organs (See FIGURE). In a Phase ½ study using DeltaRex-G for chemotherapyresistant Stage 4 pancreatic cancer, DeltaRex-G induced 100% tumor control rate, median overall survival of 9.3 months, 28.6% one-year survival rate, with minimal, if any, systemic toxicity (Chawla et al., Molecular Therapy Oncolytics, March 2019). Long term follow-up of 99 study participants showed that DeltaRex-G induced greater than 10-year survival in certain patients with chemo-resistant Stage 4 pancreatic cancer, osteosarcoma, malignant peripheral nerve sheath tumor (mPNST), breast cancer and B-cell lymphoma, and could prove to be more effective when combined with other cancer therapy/immunotherapy (Liu et al., Clinics in Oncology, 2021). Here, we report on a USFDA Emergency Use Authorized Treatment Protocol using DeltaRex-G in combination with Autologous Natural Killer Cells and low dose chemotherapy as Salvage Therapy for a patient with Stage 4 pancreatic cancer.

Study Design: Primary objective: To determine duration of survival. Secondary objective: To evaluate disease control, best overall response and the incidence of treatment-related adverse events. Exploratory objective: To correlate molecular residual disease (MRD) with treatment outcome parameters. Patient and Methods: A 40 year old white female with intractable Stage 4 pancreatic adenocarcinoma will receive DeltaRex-G 1-3×Well cfu/dose three times a week, Autologous Natural Killer Cells 4×10⁹ cells/dose every 3 weeks, Bevacizumab IV 300 mg every 2 weeks, Capecitabine p.o. 1500 mg/day×14 days followed by a one-week rest period, and Cisplatin IV 25 mg every 3 weeks until clinical disease progression or unacceptable toxicity

Statistical Analysis Plan: Descriptive statistics for demographics and incidence of individual treatment-related adverse events will be employed. Kaplan Meier plots for progression-free survival and overall survival will be conducted.

1. A method of treating cancer in an individual, the method comprising administering to the patient:
i) an anti-cancer agent comprising a binding peptide configured to bind one or more signature (SIG) elements of a tumor; and,
ii) at least one anti-cancer treatment comprising cancer immunotherapy.

2. The method of claim 1, wherein the anti-cancer agent comprises a cyclin G1 inhibitor.

3. The method of claim 2, wherein the cyclin G1 inhibitor comprises a dominant negative cyclin G1 mutant.

4. The method of claim 2, wherein the cyclin G1 inhibitor comprises a truncated form of the cyclin G1 protein.

5. The method of any one of claim 4, wherein the truncated form of cyclin G1 protein lacks the ubiquitinated N-terminus and the α1 and α2 helical segments.

6. The method of claim 4, wherein the truncated form of the cyclin G1 protein lacks amino acids 1-86.

7. The method of claim 4, wherein the truncated form of the cyclin G1 protein consists of amino acids 87-295 of cyclin G1.

8. The method of claim 2, wherein the cyclin G1 inhibitor comprises dnG1.

9. The method of claim 2, wherein the anti-cancer agent comprises a nucleic acid molecule encoding the cyclin G1 inhibitor.

10. The method of claim 9, wherein the nucleic acid molecule is a viral vector.

11. The method of claim 1, wherein the anti-cancer agent targets tumor associated fibroblasts (TAFs) or tumor associated macrophages (TAMS).

12. The method of claim 1, wherein the binding peptide comprises a polypeptide sequence having at least 80% sequence identity to a polypeptide selected from Trp-Arg-Glu-Pro-Ser-Phe-Met-Ala-Leu-Ser (SEQ ID NO:1) or Trp-Arg-Glu-Pro-Gly-Arg-Met-Glu-Leu-Asn (SEQ ID NO:2).

13. The method of claim 1, wherein the binding peptide is displayed on the surface of a nanoparticle, or a viral or pseudoviral particle.

14. The method of claim 1, wherein the biding peptide binds a SIG element present in an extracellular matrix protein of the tumor.

15. The method of claim 14, wherein the extracellular matrix protein is collagen.

16. The method of claim 1, wherein the binding peptide is configured to bind a Gly-Xxx-Pro/Hyp-Ala-Xxx-Pro/Hyp-Gly-Xxx-Pro/Hyp (SEQ ID NO:3) polypeptide sequence that is exposed on a tumor, wherein Xxx is an amino acid other than Gly, Pro, or Hyp.

17. The method of claim 1, wherein the cancer immunotherapy comprises administering immune cells to the individual.

18. The method of claim 17, wherein the immune cells are selected from NK cells and T cells.

19. The method of claim 1, wherein the cancer immunotherapy comprises administering immune checkpoint inhibitors or monoclonal antibodies.

20. The method of claim 1, wherein the anti-cancer agent comprises a thymidine kinase (TK) gene.

21. The method of claim 20, wherein cancer immunotherapy comprises administering a cytocidal compound that is activated by thymidine kinase.

22. The method of claim 21, wherein the cytocidal compound is a synthetic nucleoside analogue.

23. The method of claim 21, wherein the cytocidal compound is acyclovir or valacyclovir.

24. The method of claim 1, wherein the anti-cancer agent and the anti-cancer treatment are administered to the individual at separate times.

25. The method of claim 1, wherein the anti-cancer agent is administered to the individual at a first time, and the anti-cancer treatment is administered to the individual at a later second time.

26. The method of claim 25, wherein the second time is at least 15 minutes, at least 30 minutes, at least 1 hour, at least 12 hours, at least 24 hours, at least 2 days, at least 3 days, at least 4 days, at least 5 days, at least one week, at least two weeks, or at least one month, after the first time.

27. The method of claim 1, wherein the anti-cancer agent is DeltaRex-G or Delta-RexGT.


SEQUENCE LISTING (3 sequences)

SEQ ID NO: 1 - protein, 10 aa, Homo sapiens
WREPSFMALS

SEQ ID NO: 2 - protein, 10 aa, Homo sapiens
WREPGRMELN

SEQ ID NO: 3 - protein, 9 aa, Synthetic construct
  VAR_SEQ 2: note = X represents any amino acid other than G, P, or O (hydroxyproline)
  VAR_SEQ 5: note = X represents any amino acid other than G, P, or O (hydroxyproline)
  VAR_SEQ 8: note = X represents any amino acid other than G, P, or O (hydroxyproline)
  VAR_SEQ 6: note = X is P or O
  VAR_SEQ 9: note = X is P or O
GXPAXXGXX